(19) United States
(12) Patent Application Publication
(10) Pub. No.: US 2021/0171579 A1
(43) Pub. Date: Jun. 10, 2021

(54) INHIBITOR USING PLANT CYCLOPEPTIDE AS EFFECTIVE COMPONENT FOR LIPID METABOLIC ABNORMALITIES IN CANCER CELLS AND USES THEREOF

(71) Applicant: CHINA PHARMACEUTICAL UNIVERSITY, Nanjing (CN)

(72) Inventors: Ninghua TAN, Nanjing (CN); Yurong WANG, Nanjing (CN); Lihua SONG, Nanjing (CN)

(21) Appl. No.: 16/769,872

(22) PCT Filed: May 14, 2018
(86) PCT No.: PCT/CN2018/086727

§ 371 Date: Jun. 4, 2020

(30) Foreign Application Priority Data
Dec. 5, 2017 (CN) 201711265478.7

(51) Int. Cl. C07K 9/00; C07K 7/64; A61P 35/00

(52) U.S. Cl. CPC C07K 9/006; A61P 35/00; C07K 7/64

(57) ABSTRACT

The present disclosure discloses an inhibitor for abnormal lipid metabolism of tumor cells with a plant cyclopeptide as an effective component, and discloses application of the inhibitor to preparation of drugs for inhibiting abnormal lipid metabolism of the tumor cells, which specifically can be applied to preparation of drugs for treating and preventing abnormal lipid metabolism related cancers including liver cancer, colon cancer, rectal cancer and prostatic cancer. The inhibitor for abnormal lipid metabolism of the cells of the present disclosure can effectively inhibit abnormal lipid metabolism of the tumor cells. Its effective component, the plant cyclopeptide, is wide in source and mature in extraction technology. The inhibitor has diversified dosage forms and medication ways and has a wide clinical application prospect.

### TECHNICAL FIELD

The present disclosure belongs to the field of tumor drugs, and in particular relates to an inhibitor for abnormal lipid metabolism of tumor cells with a plant cyclopeptide as an effective component as well as a preparation method and application thereof.

### BACKGROUND ART

Lipids are one of three major nutrients, and besides being closely related to energy supply and storage, have two effects: first, the lipids are main construction molecules of cells, lipid molecules include phospholipids (glyceryl phosphatide, sphingomyelin, etc.) and cholesterol, which are main components of cell membranes, and the change of lipid metabolism directly affects cell membrane synthesis and cell proliferation; and second, the lipids are important active molecules in life activities of the cells, the lipid molecules and their metabolic intermediates can participate in multi-signal transduction of the cells, inflammations and vascular regulation, etc., and the lipids are closely related to cell proliferation, cell adhesion, movement, etc.

Abnormal lipid metabolism of tumors is an important constituent part of tumor metabolism reprogramming. A normal human body provides the lipids mainly relying on blood flow, but a lipid synthesis process of tumor cells starts de novo. Normal cells are powered mainly by aerobic oxidative phosphorylation of glucose and powered mainly by anaerobic glycolysis in an anaerobic environment. However, the tumor cells are obviously different, and still actively take in the glucose and conduct glycolysis even in the presence of adequate oxygen supply while generating a lot of lactic acids. The most important reason why the tumor cells adopt a metabolism way of aerobic glycolysis is that aerobic glycolysis products can promote uptake of nutrient substances by the tumor cells and synthesize biomacromolecules (such as nucleosides, amino acids and the lipids). Many regulatory factors in the lipid synthesis process are highly expressed in the tumor cells, but lowly expressed or not expressed in corresponding normal tissue cells. Therefore, targeting lipid synthesis has good selectivity, has more advantages and development prospects, is a research hotspot of antineoplastic antimetabolie drugs in recent years, and is widely concerned by science researchers. But there are no drugs targeting abnormal lipid metabolism of the tumors in current markets.

Rubiaceae-type cyclopeptides (RAs) are particular to rubiaceae plants, commonly exist in rubia plants, and are a class of bicyclic homocyclic hexapeptide compounds, which are hexapeptide formed mainly by connecting a D-type α-alanine, an L-type α-alanine, three L-type N-substituted α-tyrosines and one other L-type encoded α-amino acid through peptide chains, six amino acids are condensed into an eighteen-membered ring, and a benzene ring between two adjacent tyrosines is connected through an oxygen bridge to form a fourteen-membered ring with relatively large tension. The RAs have attracted much attention because of their novel bicyclic structures and significant in-vivo and in-vitro antineoplastic activity. But in the prior art, there are no reports on inhibition to abnormal lipid metabolism of the tumors by the RAs.

### SUMMARY OF THE INVENTION

The present disclosure is directed to provide an inhibitor acting on abnormal lipid metabolism of tumor cells with a rubiaceae-type cyclopeptide compound as an effective component and application thereof.

Technical Solutions: an effective component of the inhibitor for abnormal lipid metabolism of tumors of the present disclosure is a plant cyclopeptide.

Further, the plant cyclopeptide is a rubiaceae-type cyclopeptide.

Furtherer, the rubiaceae-type cyclopeptide is an RA-V or an RA-XII shown in the following structural formulas.

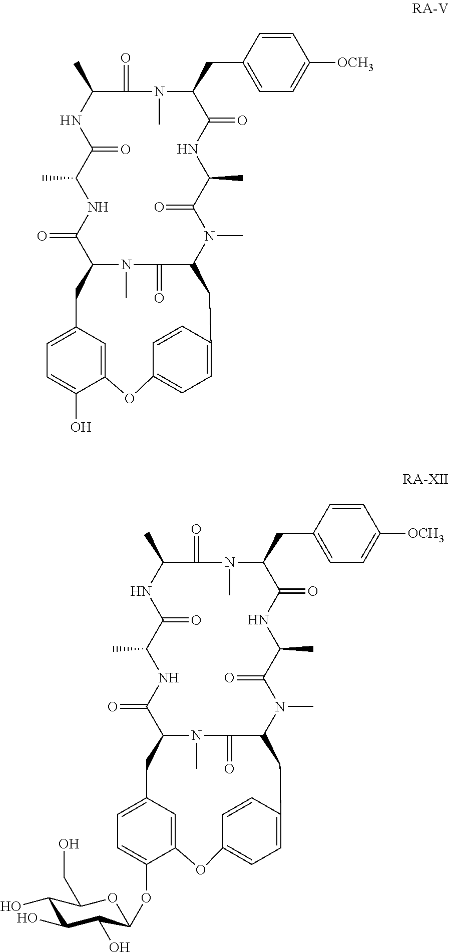

The inhibitor may be any dosage form acceptable in pharmacotherapeutics, and its dosage is any dosage acceptable in pharmacotherapeutics.

Application of the above inhibitor to preparation of drugs for treating and preventing diseases capable of benefiting from abnormal lipid metabolism of cells is also within the protection scope of the present disclosure. The application includes but is not limited to application to inhibition of the diseases benefiting from abnormal lipid metabolism of the tumor cells.

For a preparation method of the above rubiaceae-type cyclopeptides (RAs), reference is made to the China invention patent CN 201410445325.0.

Beneficial Effects: the inhibitor for lipid metabolism of the present disclosure can effectively inhibit rapid proliferation and abnormal lipid metabolism of the tumor cells, its effective component, the plant cyclopeptide, is wide in source and mature in extraction technology, and the inhibitor has diversified dosage forms and medication ways, can be applied to treatment of related cancers and has a wide clinical application prospect.

### BRIEF DESCRIPTION OF THE DRAWINGS

FIG. 1 is a growth inhibition effect of a rubiaceae-type cyclopeptide RA-XII on human liver cancer cells;

FIG. 2 is influences of rubiaceae-type cyclopeptides RA-V and RA-XII on expression of indexes related to lipid metabolism of human liver cancer cells;

FIG. 3 is inhibition of formation of lipid droplets in human liver cancer cells by a rubiaceae-type cyclopeptide RA-XII;

FIG. 4 is an in-vivo antineoplastic test of a rubiaceae-type cyclopeptide RA-XII, which adopts human liver cancer cells to establish a nude mouse in-vivo transplanted tumor model, with (1) an inhibition effect of the RA-XII on tumor growth; (2) influences of the RA-XII on weights of nude mice; and (3) subcutaneously transplanted tumors of the nude mice;

FIG. 5 is inhibition of expression of indexes related to lipid metabolism in tissues of subcutaneously transplanted tumors of nude mice by a rubiaceae-type cyclopeptide RA-XII; and

FIG. 6 is inhibition of generation of lipid droplets in tissues of subcutaneously transplanted tumors of nude mice by a rubiaceae-type cyclopeptide RA-XII.

### DETAILED DESCRIPTION OF THE INVENTION

The substantial content of the present disclosure is further illustrated with specific embodiments below in combination with accompanying drawings, but the present disclosure is not limited to this. Improvements on the present disclosure according to the substance of the present disclosure all belong to the scope of the present disclosure.

A preparation method of the rubiaceae-type cyclopeptides (RAs) below is the same as the China invention patent CN 201410445325.0.

Experimental Methods: cultivated tumor cells are treated by the RAs, and their cell proliferation inhibition activity is detected by adopting an SRB method, confirming that the compounds can inhibit abnormal proliferation of the tumor cells; and contents of cholesterol, triglycerides, low-density lipoproteins and high-density lipoproteins in the cells are detected by commercial kits, and contents of lipid droplets in the tumor cells are detected by Oil Red 0 staining, confirming that the compounds inhibit abnormal lipid metabolism of the tumor cells. In-vivo antineoplastic activity and activity of inhibiting abnormal lipid metabolism of tumors of the RAs are verified by establishing a nude mouse subcutaneously transplanted tumor model with liver cancer cells.

### Embodiment 1

A growth inhibition effect of an RA-XII on human liver cancer cells:

Cell viability is determined by adopting an SRB method. An overnight human liver cancer HepG2 cell line (purchased from the Cell Bank of Committee on Type Culture Collection of Chinese Academy of Sciences) is cultivated by a 10% FBS medium. Pancreatin is added for digestion to form a cell suspension, which is inoculated onto a 96-well plate according to an appropriate concentration at 100 μl/well. Cultivation is conducted in a CO₂ incubator for 24 h until cells adhere to walls completely. Different final concentrations of RA-XII are added, after acting for 24 h, 50 μl of a 4° C. pre-cooled TCA solution (30%, w/v) is added into each well for fixing of the cells, and a final concentration of the TCA solution is 10%. The 96-well plate is transferred into a 4° C. refrigerator for fixing for 1 h after still standing for 5 min. The 96-well plate is taken out, washed 5 times with deionized water, and aired at a room temperature. Staining: after the 96-well plate is aired at the room temperature, 70 μl of 0.4% (w/v) SRB staining solution (purchased from sigma, and prepared from 1% acetic acid) is added into each well; after staining for 30 min, the staining solution is poured out; and the 96-well plate is washed 4 times with 1% (v/v) acetic acid to remove uncombined stains, and aired at the room temperature. Stains combined with cell proteins are dissolved with 100 μl of unbuffered Tris-base solution (10 mM, pH=10.5). Oscillation is conducted for 20 min on a horizontal shaker. Optical densities are determined at 540 nm by adopting a microplate reader.

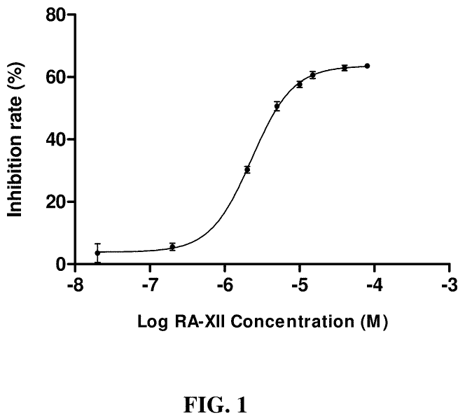

Test results are shown in FIG. 1. The results show that the RA-XII inhibits rapid proliferation of tumor cells.

### Embodiment 2

Influences of an RA-V and an RA-XII on expression of indexes related to lipid metabolism of human liver cancer cells:

An overnight human liver cancer HepG2 cell line is cultivated by a 10% FBS medium. Pancreatin is added for digestion to form a cell suspension, which is inoculated onto a 6-well plate according to an appropriate concentration. After 24 h, the RA-V or the RA-XII is added for treatment for 24 h. Digestion is conducted with the pancreatin. Centrifugation is conducted for 5 to 10 min at a room temperature and 2000 rpm, and cells are collected. The cells are resuspended once with pre-cooled 1×PBS (4° C.), centrifuged for 5 to 10 min at 2000 rpm, and washed. By adopting commercial kits, contents of total cholesterol and triglycerides in the cells are detected through a single reagent GPO-PAP method respectively, and contents of low-density lipoproteins and high-density lipoproteins in the cells are detected through a double-reagent direct method (the commercial kits are purchased from the Nanjing Jiancheng Bioengineering Institute, with article numbers of A111-1, A110-1, A113-1 and A112-1 respectively).

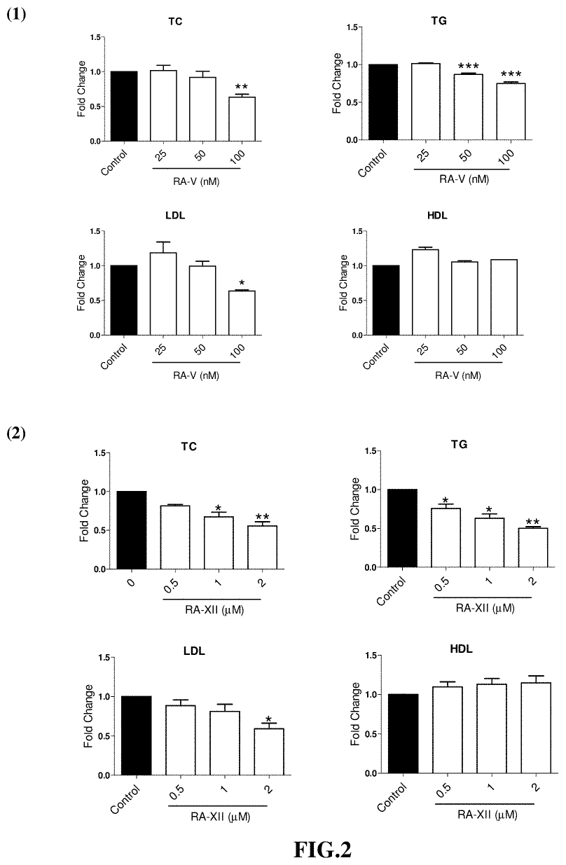

Test results are shown in FIG. 2. The results show that the RA-V and the RA-XII obviously inhibit expression of the indexes of the total cholesterol, the triglycerides and the low-density lipoproteins related to lipid metabolism in the tumor cells, but have no obvious influence on the high-density lipoproteins beneficial to a human body.

### Embodiment 3

Inhibition of formation of lipid droplets in human liver cancer cells by an RA-XII:

0.5 g of pre-ground and pre-crushed dried Oil Red powder (purchased from Sigma Company) is weighed, and dissolved in a small amount of isopropanol. Then isopropanol is added to reach 100 ml. A solution is sealed in a brown bottle (or packaged with tin foil paper for avoiding light) for preservation at 4° C. as a storage solution, which may be preserved for a long time. When using, 6 ml of the solution is taken, and 4 ml of tri-distilled water is added to be evenly mixed. The mixed solution is filtered by qualitative filter paper into a working solution, which is used up within a few hours after diluted. An overnight human liver cancer HepG2 cell line is cultivated by a 10% FBS medium. Pancreatin is added for digestion to form a cell suspension, which is inoculated onto a 24-well plate according to an appropriate concentration. After 24 h, the RA-XII is added for treatment for 24 h. A cultivation solution is poured out carefully and slowly. Fixing is then conducted with 4% paraformaldehyde for 30 min. Staining is conducted with an Oil Red working solution for about 30 min at the room temperature. Washing is conducted with PBS twice. Pictures are taken under a microscope.

Test results are shown in FIG. 3. The results show that the RA-XII can obviously reduce the number of the lipid droplets in the tumor cells.

### Embodiment 4

An in-vivo antineoplastic test of an RA-XII:

HepG2 is diluted into 1×10/mL with a serum-free MEM. 100 μL of cell suspension is taken and inoculated to left armpits of BABL/c nude mice subcutaneously, and grows for 7 days to form a tumor-bearing mouse model. Inoculated and well-grown tumor-bearing mice are grouped at random, and dosed with the RA-XII once through caudal veins every other day. While dosing, sizes of tumors are measured with a vernier caliper (a tumor volume=a long diameter×a short diameter²/2), and weights of the nude mice are recorded and counted. The used animals are killed after dosed for 21 days, and tumors are peeled and photographed.

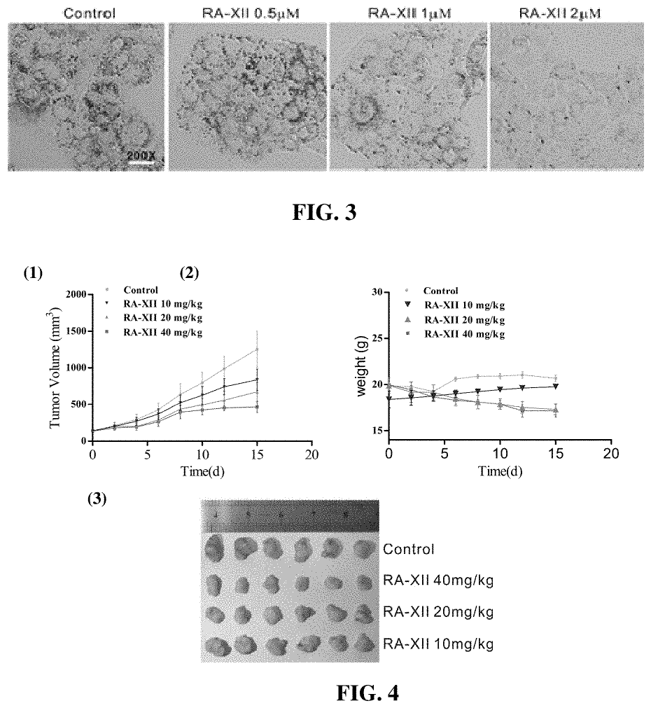

Test results are shown in FIG. 4. The results show that the RA-XII can obviously reduce a growth speed of the subcutaneously transplanted tumors of the nude mice, and has no obvious influence on the weights of the nude mice.

### Embodiment 5

Inhibition of expression of indexes related to lipid metabolism in tissues of subcutaneously transplanted tumors of nude mice by an RA-XII:

Tissues of the tumors obtained in Embodiment 4 are taken and lysed with an RIPA lysate. Then contents of cholesterol, triglycerides, low-density lipoproteins and high-density lipoproteins are detected by adopting commercial kits (specific methods are the same as Embodiment 2).

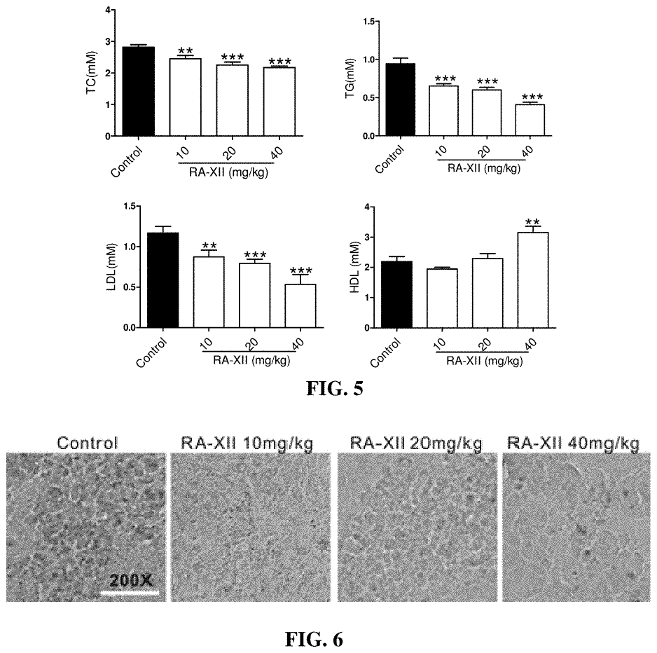

Test results are shown in FIG. 5. The results show that the RA-XII can obviously inhibit expression of total cholesterol, the triglycerides and the low-density lipoproteins in the tissues of the subcutaneously transplanted tumors of the nude mice, but has no obvious influence on the high-density lipoproteins beneficial to a human body.

### Embodiment 6

Inhibition of formation of lipid droplets in tissues of subcutaneously transplanted tumors of nude mice by an RA-XII:

Tissues of the tumors obtained in Embodiment 4 are taken and embedded by OCT and then rapidly cooled. Cryostat serial sectioning is conducted with a thickness being 20 μm. Drying is conducted for 30 min at a room temperature. Fixing is conducted with 4% paraformaldehyde for 30 min. Staining is conducted with an Oil Red working solution for about 30 min at the room temperature. Washing is conducted with PBS twice. Sections are sealed with a glycerin jelly. Overnight drying is conducted, and pictures are taken under a microscope.

Test results are shown in FIG. 6. The results show that the RA-XII can obviously reduce the number of the lipid droplets in the tissues of the subcutaneously transplanted tumors of the nude mice.

What is claimed is:

1. An inhibitor for abnormal lipid metabolism of tumor cells, wherein an effective component of the inhibitor is a plant cyclopeptide.

2. The inhibitor according to claim 1, wherein an effective component of the inhibitor is a rubiaceae-type cyclopeptide.

3. The inhibitor according to claim 2, wherein an effective component of the inhibitor is a rubiaceae-type cyclopeptide RA-V or RA-XII shown in the following structural formulas.

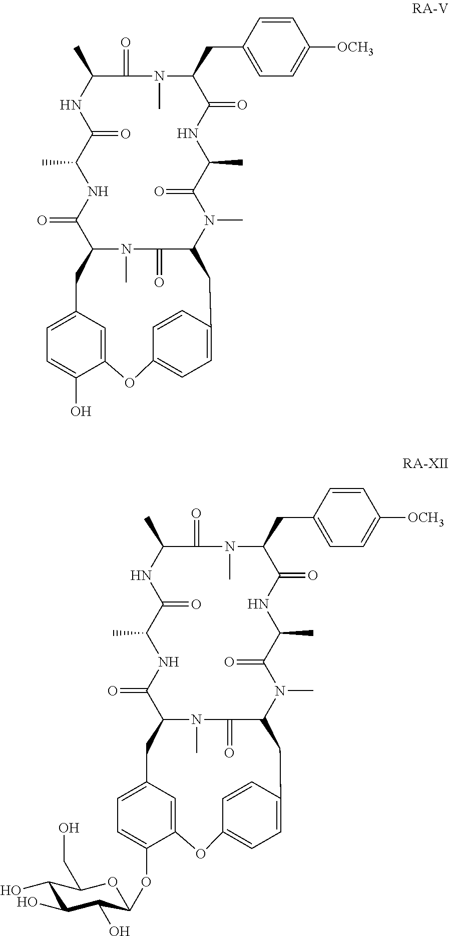

4. Application of the inhibitor according to claim 1 to preparation of drugs for treating and preventing diseases capable of benefiting from abnormal lipid metabolism of cells.

5. Application of the inhibitor according to claim 1 to preparation of drugs for targeted inhibition of abnormal lipid metabolism of tumors.

6. The application according to claim 5, wherein the tumors comprise liver cancer, colon cancer, rectal cancer, lung cancer, breast cancer and prostatic cancer.

7. The application according to claim 5, wherein the drugs inhibit abnormal proliferation of tumor cells by inhibiting abnormal lipid metabolism of the tumor cells.
